(19) United States
(12) Patent Application Publication
(10) Pub. No.: US 2005/0172975 A1
(43) Pub. Date: Aug. 11, 2005

(54) Point structured fitness system

(76) Inventor: Ed Downs, Pembroke Pines, FL (US)

Correspondence Address: Ruben Alcoba, Esq., 17347 SW 20th Ct, Miramar, FL, 33029, US

(21) Appl. No.: 10/774,730

(22) Filed: Feb. 9, 2004

(51) Int. Cl. A61B 19/00; A63B 69/00; G09B 19/00; G09B 9/00

(52) U.S. Cl. 128/898; 434/247

(57) ABSTRACT

The present invention is directed to a method of training an exercise participant that satisfies the following needs: it allows any participant to gradually increase his level of exertion when exercising; it motivates a participant to be physically active based on a rewards system; it prevents a participant from over-exerting himself; and it provides a participant with an exercise system that can be used with other dietary point systems. The method of exercising an exercise participant comprises the steps of first providing a participant with an activity chart so that the participant can determine a fitness level, then assigning a daily cardio exercise fitness number to the participant that is based on the participant's fitness level, wherein the cardio exercise fitness number will determine what cardio exercises the participant is to perform, then assigning a daily resistance exercise fitness number to the participant that is based on the participant's fitness level, wherein the resistance exercise fitness number will determine what resistance exercises the participant is to perform, then instructing the participant to perform cardio exercises a predetermined amount of days per week, the number of days being determined by the participant's fitness level, and lastly, instructing the participant to perform resistance exercises a predetermined amount of days per week, the number of days being determined by the participant's fitness level.

### BACKGROUND

After years of training individuals, Mr. Ed Downs realized that there was a need to design an exercise program that could be followed by all individuals, regardless of their fitness level. He further realized that the exercise program had to be easily understood. He realized that by giving an exercise routine a point system, individuals were more likely than not to follow the exercise routine. It became apparent to him, that when he informed individuals that a numerical goal was to be reached for each exercise session, individuals would strive to meet the goal. The problem he faced when devising this system was twofold. First, he had to help individuals determine their own fitness level, ego aside. Second, he had to make a point system that was geared toward that fitness level.

A disadvantage of current workout routines is that they do not take concern in recognizing the fitness level of an individual when preparing routines for the individual. Mr. Down's, on the other hand, believes that giving an individual the option to recognize his own fitness level, based on a fitness level chart, the individual can prepare his own routine using the ED DOWNS 3-2-1 FITNESS SYSTEM^{SM} (hereinafter referred as “3-2-1 FS”).

A problem that Mr. Downs recognizes when training individuals is that he is occasionally presented with individuals that do not recognize their own fitness level. This problem causes individuals to either injure themselves within the first week of following an exercise program or to burn themselves out. The problem is not one of intelligence, but one that relates to the psyche of the individuals. The problem caused him to realize that he had to devise a system that would allow individuals to truthfully decide their fitness level prior to assigning them an exercise program.

For this reason, Mr. Downs developed the 3-2-1 FS. The 3-2-1 FS is designed to allow any individual, regardless of his fitness level, to chose his own workout routine. He believes that an individual, given the proper tools (the 3-2-1 FS) and education, will choose the right exercise routine.

Mr. Down's believes that this exercise point system should be a compliment to any dietary plan.

An object of this invention is that it allows a sedimentary person to intelligently choose and commence an exercise routine.

A further object of this invention is to allow any individual to gradually increase their level of exertion when exercising.

Yet, another object of this invention is to motivate individuals to become physically active based on a rewards system.

Another object of this invention is to prevent individuals from over-exerting themselves: this occurs at all fitness levels.

A further object of this invention is to provide an exercise point system that can be used with other dietary point systems, e.g., WEIGHTWATCHERS.

For the forgoing reasons, there is a need for a point structured exercise routine that can be followed by a beginner as well as an expert.

### SUMMARY

The 3-2-1 FS is a structured fitness program designed to help a participant follow a flexible method of mapping out a daily fitness routine. The program can be used alone or can coincide with any dietary plan. The program allows a participant, regardless of whether he is at a beginner, intermediate or advance fitness level, to attain fitness goal.

The fitness program uses a point system to keep track of the participant's daily fitness activity. The program has assigned various exercises and activities with a number value between 3, 2, or 1. There are 5 categories of exercises to choose from: Cardiovascular, Lower Body, Upper Body, Abdominals, and Daily Activities.

Depending on the participant's fitness level, the participant is responsible for performing a certain number of exercises per day, the total point value of the exercises performed per day give the participant either his cardio exercise fitness number or his resistance fitness number. There are three main fitness levels and three sublevels within each main fitness level. As the participant is promoted from level to level, the cardio and the resistance fitness number increases.

The participant is allowed to choose any number of exercises as long as his cardio exercise fitness number or resistance fitness number is reached at the end of the day. The participant's fitness level will determine how many times a week the participant will perform a cardio or a resistance exercise.

The participant is evaluated every six weeks to determine whether the fitness level of the participant is to be increased. The program takes care of not raising the participant's fitness level at an accelerated rate, thereby maximizing the effectiveness of the fitness program.

The present invention is directed to a method of training an exercise participant that satisfies the following needs: it allows any participant to gradually increase his level of exertion when exercising; it motivates a participant to be physically active based on a rewards system; it prevents a participant from over-exerting himself; and it provides a participant with an exercise system that can be used with other dietary point systems. The method of exercising an exercise participant comprises the steps of first providing a participant with an activity chart so that the participant can determine a fitness level, then assigning a daily cardio exercise fitness number to the participant that is based on the participant's fitness level, wherein the cardio exercise fitness number will determine what cardio exercises the participant is to perform, then assigning a daily resistance exercise fitness number to the participant that is based on the participant's fitness level, wherein the resistance exercise fitness number will determine what resistance exercises the participant is to perform, then instructing the participant to perform cardio exercises a predetermined amount of days per week, the number of days being determined by the participant's fitness level, and lastly, instructing the participant to perform resistance exercises a predetermined amount of days per week, the number of days being determined by the participant's fitness level.

### DRAWINGS

These and other features, aspects, and advantages of the present invention will become better understood with regard to the following description, appended claims, and accompanying drawings where:

FIG. 1 is a monthly fitness calendar example for a participant who is a beginner;

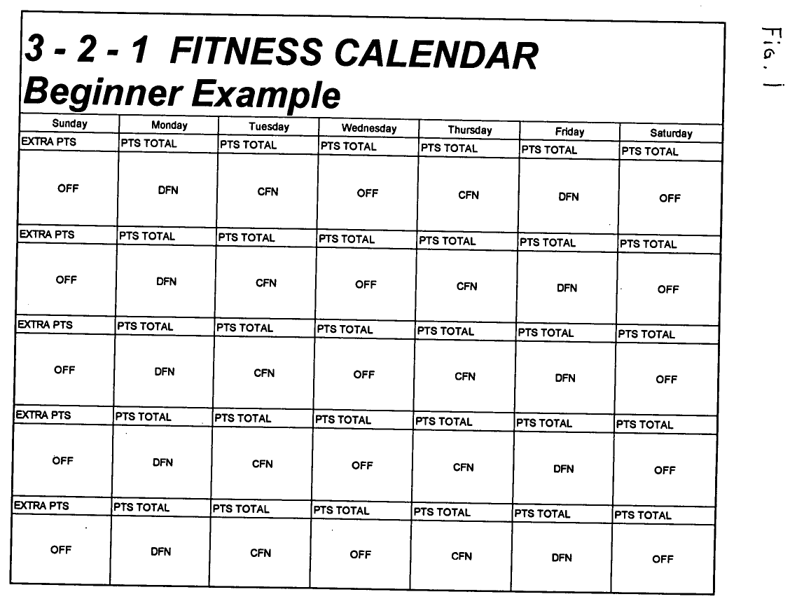

FIG. 2 is a monthly fitness calendar example for a participant who is an intermediate; and

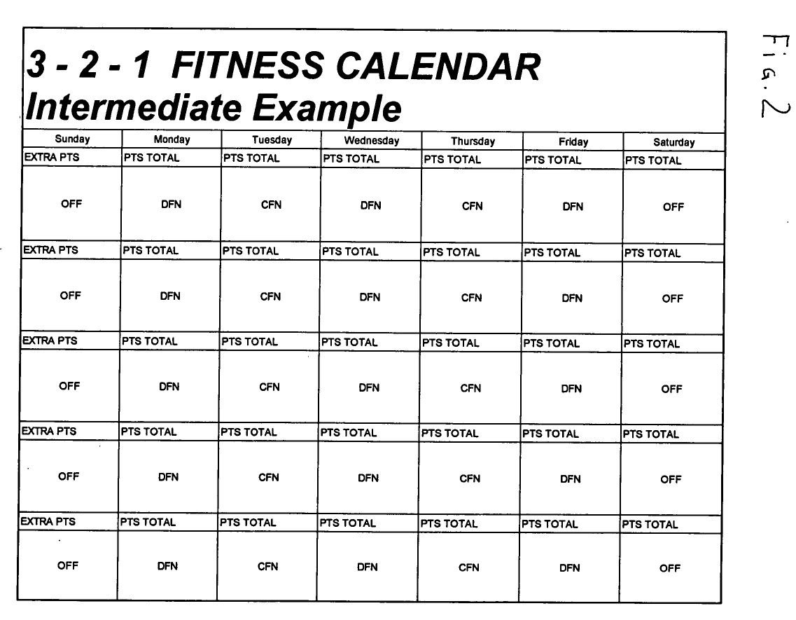

FIG. 3 is a monthly fitness calendar example for a participant who is advanced.

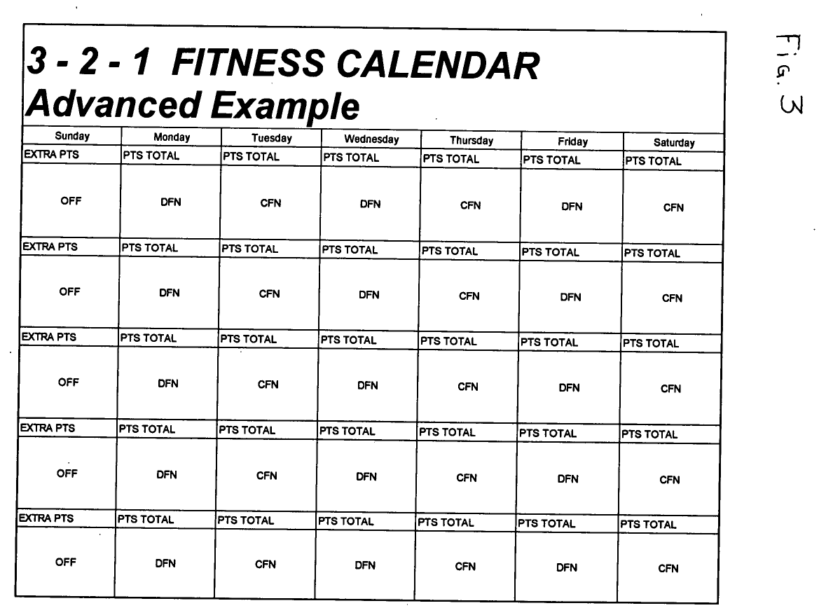

### DEFINITIONS

Before proceeding with a detailed description of the preferred embodiments, definitions or explanations of selected terms as employed in this specification and in the claims are set forth as follows:

How to determine a participant's fitness level: the fitness level is determined by the times a participant exercises per week. e.g.

| Workouts each week | Activity level | Category |
|---|---|---|
| 0-1 | Sedentary | Beginner |
| 2-3 | Moderately Active | Intermediate |
| 4 or more | Active | Advanced |

There are 3 categories of Fitness; Beginner (B), Intermediate (I), and Advance (A). Each of these categories has three levels of fitness, level 1 being the easiest and level 3 requiring the most effort/skill. Each Level is assigned a Daily Resistance Fitness Number (DRFN—the sum of all resistance exercise point values earned each day). It is also assigned a Daily Cardio Fitness Number (DCFN—the sum of cardio exercise point values earned each cardiovascular day).

TABLE 1
Fitness Level

| Categories | Levels | DRFN | DCFN |
|---|---|---|---|
| Beginner | B1 | 23 or less | 6 |
| | B2 | 24-25 | 6 |
| | B3 | 26-27 | 6 |
| Intermediate | I1 | 28-29 | 9 |
| | I2 | 30-31 | 9 |
| | I3 | 32-33 | 9 |
| Advanced | A1 | 34-35 | 12 |
| | A2 | 36-37 | 12 |
| | A3 | 38-39 | 12 |

M.I.=Muscles Involved

resistance exercise point value (REPV)=(M. I.×repetition of exercise)/20

cardio exercise point value (CEPV)=(M.I.×minutes exercised)/20

3-2-1 Fitness Formula: is a formula used to calculate the Points given for the various exercises and activites that range from 60-75% exertion. The 3-2-1 Fitness Formula=(Muscles Involved×Duration (min.) or Repetitions)/E.D. Constant The Muscles Involved represents the number of major muscle groups recruited in the exercise or activity.

The Duration or Repetitions is the time spent on the activity or the Repetitions performed on the exercise.

The E.D. Constant at the bottom of the equation is a fixed number of 20.

Multiplying the Muscles Involved by the Duration or Repetitions and then dividing that number by the E.D. Constant will give you the exercise fitness point value for each exercise.

For Example:

A Basic ½ Squat uses 4 major muscle groups (Glutes, Quadriceps, Hamstrings and Adductors). If you were to do a set of squats for 15 repetitions the equation would be as follows: (4×15 reps)/20=3

Pre-calculated point values for the five categories of exercises to choose from are as follows:

CHART A
3-2-1 Cardio Chart:

| Exercise | Duration/Pace (m/hr) | Distance | Points |
|---|---|---|---|
| Walking | 24-30 min/2.0-2.5 | 1 mile | 6 |
| Walk/Brisk Walk | 20-24 min/2.5-3.0 | 1 mile | 6 |
| Brisk Walk | 17-20 min/3.0-3.5 | 1 mile | 6 |
| Jog/Walk | 15-17 min/3.5-4.0 | 1 mile | 6 |
| Jog | 13-15 min/4.0-4.5 | 1 mile | 6 |
| Jog/Run | 12-13 min/4.5-5.0 | 1 mile | 6 |
| Run | <12 min/5.0< | 1 mile | 6 |

REMEMBER the points are given based on the fitness level of the individual. For example an unfit person walking for 30 minutes is exerting the same amount of energy and effort as a very fit person running 30 minutes. On the other hand, if the Very fit person chooses to walk, they Do Not get there 6 points shown above.

Using the Minute ON—Minute OFF Principle. If you choose to do one of the interval cardio training routines above (e.g. walk/brisk walk), you would walk for a minute and then brisk walk for one minute, alternating.

CHART B
Lower Body Chart:

| Exercise | Sets/Repetitions | Points |
|---|---|---|
| Squats (full, half, and quarter) | 1/15 | 3.0 |
| Plies | 1/15 | 2.5 |
| Leg extensions | 1/15 | 2.0 |
| Leg curls | 1/15 | 2.0 |
| Standing hip extensions | 1/15 | 1.5 |
| All Fours —hip extensions | 1/15 | 1.5 |
| Glute kickbacks | 1/15 | 1.5 |
| Calve raises | 1/20 | 1.5 |
| Inner thigh raises | 1/15 | 1.5 |
| Scissors | 1/15 | 1.5 |
| Outer thigh raises | 1/15 | 1.5 |
| Outer thigh raises standing | 1/15 | 1.5 |

CHART C
Upper Body Exercises Chart:

| Exercise | Sets/Repetitions | Points |
|---|---|---|
| Chest Flys | 1/15 | 2.0 |
| Pushups | 1/10 | 2.0 |
| Straight arm pullovers | 1/15 | 2.0 |
| Super mans | 1/10 | 2.0 |
| Double arm row | 1/15 | 2.0 |
| Single arm row | 1/15 | 1.5* |
| Seated or standing shoulder press | 1/15 | 1.5* |
| Shoulder (deltoid) Side raises | 1/15 | 1.0 |
| Shoulder Front raises | 1/15 | 1.0 |
| Triceps kickbacks | 1/15 | 1.0 |
| Overhead triceps extensions | 1/15 | 1.0 |
| Alternating Hammer curls | 1/15 | 1.0 |
| Bicep curls | 1/15 | 1.0 |

CHART D
Abs (core) Exercises Chart:

| Exercise | Sets/Repetitions | Points |
|---|---|---|
| Standing twist (endomorphs) | 1/20 | 1.5 |
| Standing FIG. 8 (endomorphs) | 1/20 | 1.5 |
| 45 degree hold and twist | 1/20 | 1.5 |
| twist and tap | 1/20 | 1.5 |
| 45 degree FIG. 8 | 1/20 | 1.5 |
| knee to chest | 1/20 | 1.5 |
| crunches (feet on chair) | 1/20 | 1.5 |
| crossover crunches | 1/20 | 1.5 |
| | each side | |
| punching crunches | 1/10 | 1.5 |
| bicycles | 1/10 | 1.5 |
| reverse crunches bent knee | 1/10 | 1.5 |
| reverse crunches straight leg | 1/10 | 1.5 |

CHART E
Altering Daily Activities* Chart:

| Exercise | Sets/Repetitions | Points |
|---|---|---|
| Walking upstairs (Stairmill) | | |
| Chair squats (Squats) | | |
| Chair dips (Triceps Extensions) | | |
| Reaching for object on your tip toes | | |
| (calve raises) | | |
| Vacuuming (Rows) | | |
| Washing windows (Shoulder Raises) | | |
| Picking up your baby (Bicep curls and | | |
| Shoulder Press) | | |
| Baby chest press (Bench Press) | | |
| Getting clothes out of Dryer (Squats) | | |
| Changing TV channel (Walking Lunge) | | |
| Getting water out of Refrigerator | | |
| (Walking Lunge | | |

*Daily activities can earn points for the participant when the activity is performed like the exercise assigned to the activity.

### DESCRIPTION

A method of training an exercise participant that is based on a point system, comprising the steps of providing a participant with an activity chart so that the participant can determine a fitness level; assigning a daily cardio exercise fitness number to the participant that is based on the participant's fitness level, wherein the cardio exercise fitness number will determine what cardio exercises the participant is to perform; assigning a daily resistance exercise fitness number to the participant that is based on the participant's fitness level, wherein the resistance exercise fitness number will determine what resistance exercises the participant is to perform; instructing the participant to perform cardio exercises a predetermined amount of days per week, the number of days being determined by the participant's fitness level; and instructing the participant to perform resistance exercises a predetermined amount of days per week, the number of days being determined by the participant's fitness level.

In the providing the participant with an activity chart step, the participant is allowed to decide whether he is sedentary, moderately active, or active. The method of training the participant will be determined based on the physical fitness of the participant. Upon the participant deciding his level of activity, a fitness level will be assigned to the participant. Each fitness level has three sublevels, each sublevel gradually increasing the number of the cardio and the resistance exercise fitness number required per-day.

In the assigning a daily cardio exercise fitness number to the participant step, the cardio exercise fitness number is based on the participant's fitness level, and the cardio exercise fitness number will determine what cardio exercises the participant is to perform. Cardio exercises that the participant can perform are referred above in chart A. In practice, the participant will first determine the cardio exercise point value (CEPV) for each exercise performed and then add all of the CEPVs performed that day together. The participant will comply with the daily cardio exercises required when the sum of the CEPVs equals the daily cardio fitness number.

In the assigning a daily resistance exercise fitness number to the participant step, the resistance exercise fitness number is based on the participant's fitness level, and the resistance exercise fitness number will determine what resistance exercises the participant is to perform. Resistance exercises that the participant can perform are referred above in charts B-E. In practice, the participant will first determine the Resistance exercise point value (REPV) for each exercise performed and then add all of the REPVs performed that day together. The participant will comply with the daily resistance exercises required when the sum of the REPVs equals the daily resistance fitness number.

In the instructing the participant to perform cardio exercises a predetermined amount of days per week step, the number of days will be determined by the participant's fitness level. The instruction can be relayed to the participant via an actual instructor, a virtual instructor, a video, or any other medium know to convey instruction.

In the instructing the participant to perform resistance exercises a predetermined amount of days per week step, the number of days will be determined by the participant's fitness level. The instruction can be relayed to the participant via an actual instructor, a virtual instructor, a video, or any other medium know to convey instruction.

In another embodiment of this invention, the method of training an exercise participant can comprise the step of evaluating the paricipant's dietary plan and combining it with the training program of the participant. The participant will benefit most from this method of training when the method is combined with a clinically approved dietary program.

In another embodiment of the invention, the method of training an exercise participant can comprise the step of monitoring the participant's progress every six weeks and determining whether the participant is to be raised to a higher fitness level. Ideally, an instructor should determine the fitness level of the participant after six weeks, but it is possible for the participant to determine his own fitness level. If the participant decides to determine his own fitness level, it is crucial that the participant be truthful with himself, for honesty is the best way of avoiding injury.

The following example of the method of training an exercise participant, shall presume that a twenty four year old female participant has been assigned a specific weekly workout routine for her fitness level. The participant weighs 190 lbs. and is 5′6.″ Her Target weight is 140 lbs. The participant has decided that her fitness level is B2, so when she looks at the fitness chart she realizes that her DRFN is 24 and her DCFN is 6. Using the DRFN and the DCFN she assigns herself the following routine:

On Monday, Wednesday and Friday she will perform the following exercises,

| Exercise | Sets/Reps or Duration | Points |
|---|---|---|
| Walking | 30 minutes | 6 |
| ½ Squats | 3 sets/15 reps | 9 |
| Triceps Kickbacks | 3 sets/15 reps | 3 |
| Standing Ab Twist | 3 sets/15 reps | 6, |

Terry earns a total of 24 points daily after performing all of the above exercises. Therefore, Terry reaches her DRFN for the day, her day has been successful. Note, Terry might exceed the number of points for the day and bank them for another day during that week.

On Tuesday, Thursday and Saturday she will perform the following exercises,

| Exercise | Sets/Reps or Duration | Points |
|---|---|---|
| Walking | 30 minutes | 6 |

Terry earns a total of 6 points daily after performing the above exercise. Therefore, Terry reaches her DCFN for the day, her day has been successful. Note, Terry might exceed the number of points for the day and bank them for another day during that week.

The concept of banked points means that if you do more exercises than required on a certain date, you can apply does points to another day during that same week. The inventor does not recommend this approach, for it may lead to injury to the participant.

It should be remembered that this example of the exemplary workout method was intended to be illustrative, not limiting, and various changes, modifications, and/or adaptations may be made without departing from the spirit and scope of this invention.

The terms and expressions employed herein are employed as terms of description and not limitation; and thus there is no intent to exclude equivalents, but on the contrary it is intended to cover any and all equivalents that may be employed without departing from the spirit and scope of the invention.

1. A method of training an exercise participant that is based on a point system, comprising the steps of:
providing a participant with an activity chart so that the participant can determine a fitness level;
assigning a daily cardio exercise fitness number to the participant that is based on the participant's fitness level, wherein the cardio exercise fitness number will determine what cardio exercises the participant is to perform;
assigning a daily resistance exercise fitness number to the participant that is based on the participant's fitness level, wherein the resistance exercise fitness number will determine what resistance exercises the participant is to perform;
instructing the participant to perform cardio exercises a predetermined amount of days per week, the number of days being determined by the participant's fitness level; and
instructing the participant to perform resistance exercises a predetermined amount of days per week, the number of days being determined by the participant's fitness level.

2. The method of claim 1, further comprising the step of evaluating the participant's dietary plan and combining it with the training program of the participant.

3. The method of claim 2, further comprising the step of monitoring the participant's progress every six weeks and determining whether the participant is to be raised to a higher fitness level.

4. The method of claim 1, wherein each fitness level has three sublevels, each sublevel gradually increasing the number of the cardio and the resistance exercise fitness number required per-day.

5. The method of claim 4, wherein the cardio exercise fitness number is determined by first determining a CEPV of each exercise performed each day and then adding all of the CEPVs together.

6. The method of claim 5, wherein the resistance exercise fitness number is determined by first determining a REPV of each exercise performed each day and then adding all of the REPVs together.

7. The method of claim 6, further comprising the step of evaluating the participant's dietary plan and combining it with the training program of the participant.

8. The method of claim 7, further comprising the step of monitoring the participant's progress every six weeks and determining whether the participant is to be raised to a higher fitness level.

9. The method of claim 6, further comprising the step of monitoring the participant's progress every six weeks and determining whether the participant is to be raised to a higher fitness level.
